(19) United States
(12) Patent Application Publication
(10) Pub. No.: US 2016/0184133 A1
(43) Pub. Date: Jun. 30, 2016

(54) THERAPY DEVICE

(71) Applicant: CANON KABUSHIKI KAISHA, Tokyo (JP)

(72) Inventors: Takuro Miyasato, Tokyo (JP); Kazuhiko Fukutani, Yokohama-shi (JP); Yoshiko Nakamura, Kyoto-shi (JP); Takuji Oishi, Kawasaki-shi (JP); Hiroshi Abe, Kyoto-shi (JP); Ryuichi Nanaumi, Tokyo (JP); Kohtaro Umezawa, Tokyo (JP)

(21) Appl. No.: 14/973,865

(22) Filed: Dec. 18, 2015

(30) Foreign Application Priority Data
Dec. 26, 2014 (JP) 2014-265102

(51) Int. Cl. A61F 7/08; A61B 5/00

(52) U.S. Cl. CPC A61F 7/08; A61B 5/0095; A61F 2007/0095

(57) ABSTRACT

A therapy device comprises a light source; an irradiation unit configured to irradiate an object with light from the light source; a reception unit configured to receive an acoustic wave generated and propagated within the object due to the light, and output the acoustic wave as an electrical signal; a processing unit configured to acquire characteristic information of the object based on the electrical signal output from the reception unit; a temperature adjustment unit configured to be capable of changing a temperature of the object; and a temperature control unit configured to control the temperature adjustment unit based on the characteristic information.

### BACKGROUND OF THE INVENTION

1. Field of the Invention

The present invention relates to a therapy device.

2. Description of the Related Art

In ice therapy, heat therapy, and cryokinetics, injuries are prevented and treated by cooling, heating, or pressurizing an affected area. For example, Japanese Patent Application Laid-open No. 2012-187397 discloses a device that performs heat therapy such that the temperature of a patient is raised, held, and lowered in accordance with a temporal shift in temperature set in advance, based on the temperature of the patient measured with a temperature sensor.

### SUMMARY OF THE INVENTION

However, what amount of blood in the affected area contributes to the effect of therapy cannot be known from the temperature of the affected area. Therefore, as with a method described in Japanese Patent Application Laid-open No. 2012-187397, there is a possibility that a sufficient therapeutic effect is not obtained with a method of controlling the assigned temperature, depending on the measured temperature of the affected area.

In view of the problem described above, an object of the present invention is to provide a therapy device that can enhance the therapeutic effect in an affected area.

The present invention in its one aspect provides a therapy device comprising a light source; an irradiation unit configured to irradiate an object with light from the light source; a reception unit configured to receive an acoustic wave generated and propagated within the object due to the light, and output the acoustic wave as an electrical signal; a processing unit configured to acquire characteristic information of the object based on the electrical signal output from the reception unit; a temperature adjustment unit configured to be capable of changing a temperature of the object; and a temperature control unit configured to control the temperature adjustment unit based on the characteristic information.

The present invention in its another aspect provides a therapy device comprising a light source; an irradiation unit configured to irradiate an object with light from the light source; a light detection unit configured to receive light radiated from the irradiation unit and propagated within the object, and output the light as an electrical signal; a processing unit configured to acquire characteristic information of the object based on the electrical signal output from the light detection unit; a temperature adjustment unit configured to be capable of changing a temperature of the object; and a temperature control unit configured to control the temperature adjustment unit based on the characteristic information.

The present invention provides a therapy device that can enhance the therapeutic effect in an affected area.

Further features of the present invention will become apparent from the following description of exemplary embodiments with reference to the attached drawings.

### BRIEF DESCRIPTION OF THE DRAWINGS

FIG. 1 is a block diagram showing a therapy device in Embodiment 1 of the present invention;

FIG. 2 is a block diagram showing a therapy device in Embodiment 2 of the present invention;

FIG. 3 is a block diagram showing a therapy device in Embodiment 3 of the present invention; and

FIG. 4 is a diagram showing a screen of a display unit in the therapy device in Embodiment 3.

### DESCRIPTION OF THE EMBODIMENTS

An embodiment of the present invention will be described below in detail with reference to the drawings. For the same components, the same reference numerals are assigned as a principle, and descriptions are omitted. Note that the specific calculation formulae, calculation procedures, and the like described below should be changed appropriately depending on the configuration or various conditions of a device to which the invention is applied, and it is not intended to limit the scope of the invention to the description below.

A device of the present invention includes a device that utilizes the photoacoustic effect to receive acoustic waves generated within an object by irradiating the object with light (electromagnetic waves) such as near-infrared light and acquire characteristic information of the object as image data. In the case of a device utilizing the photoacoustic effect, characteristic information of an object to be acquired is generation source distribution of acoustic waves caused by light irradiation, initial sound pressure distribution within the object, light energy absorption density distribution or absorption coefficient distribution derived from initial sound pressure distribution, concentration distribution of a substance forming a tissue, or the like. The concentration distribution of a substance is, for example, oxygen saturation distribution, total hemoglobin concentration distribution, oxy/deoxy hemoglobin concentration distribution, or the like. It may be such that the degree of inflammation in an observed segment is estimated from the characteristic information, and such information is assumed as the characteristic information.

Characteristic information of an object in a plurality of positions may be acquired as a two-dimensional or three-dimensional characteristic distribution. The characteristic distribution can be generated as image data showing characteristic information of an object.

Acoustic waves referred to in the present invention are typically ultrasound waves and include elastic waves called sound waves or ultrasound waves. Acoustic waves generated by the photoacoustic effect are called photoacoustic waves or light-induced ultrasound waves. An acoustic detector (e.g., a probe) receives acoustic waves generated within an object.

### Embodiment 1

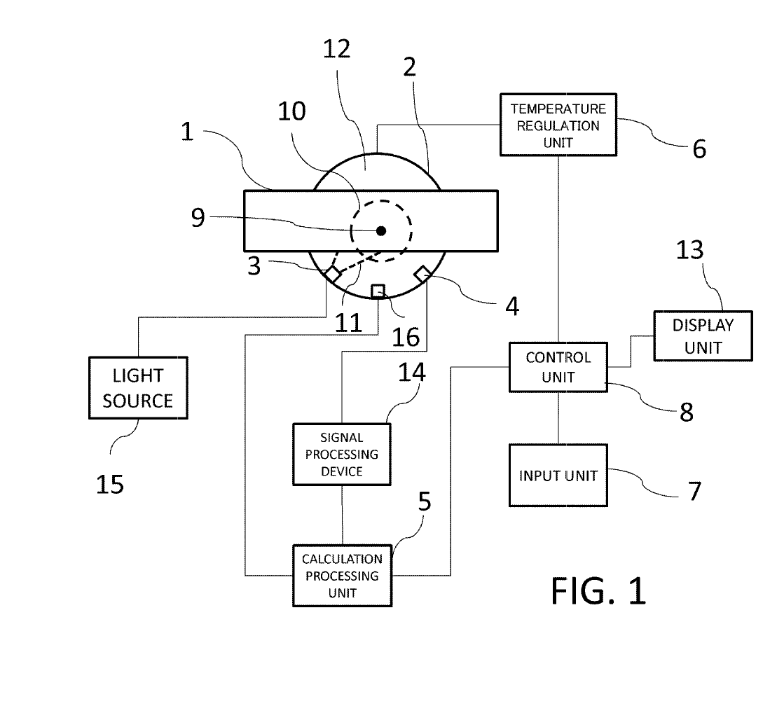

FIG. 1 is a block diagram showing Embodiment 1 of a therapy device according to an embodiment of the present invention. A therapy device 100 (hereinafter, abbreviated as “device 100”) of Embodiment 1 includes an irradiation unit 3 that irradiates an object 1 with single-wavelength pulsed light 11. Further, the device 100 includes an acoustic wave detection unit 4 (corresponding to a reception unit) with which a photoacoustic wave 10 generated by an absorber 9 within the object 1 absorbing energy of light from the irradiation unit 3 is detected, converted to an analog electrical signal, and output. Further, the device 100 includes a signal processing device 14 that receives an analog electrical signal output from the acoustic wave detection unit 4, amplifies the analog electrical signal, and generates and outputs a digital signal formed by analog/digital conversion. Further, the device 100 includes a calculation processing unit 5 (corresponding to a processing unit) that calculates characteristic information of the object 1 using a digital signal output from the signal processing device 14. Characteristic information to be obtained with the device according to the present invention includes information reflecting the amount of blood within an object. Further, the device 100 includes a water bag 2 containing a solvent 12 that is provided near the object 1 to contact the object 1 in order to change the temperature of the object 1. Further, the device 100 includes a temperature regulation unit 6 that changes the temperature of the object 1 indirectly by changing the temperature of the solvent 12. Further, the device 100 includes an input unit 7 with which an operator is capable of inputting a target value regarding characteristic information of the object 1 such as the amount of hemoglobin. The calculation processing unit 5 as a setting unit sets a target value of characteristic information of the object 1, based on the information input by the input unit 7. Further, the device 100 includes a control unit 8 that controls the temperature regulation unit 6, based on characteristic information of the object 1 obtained by the calculation processing unit 5 and a target value of characteristic information set by the calculation processing unit 5. The device 100 includes a display unit that displays obtained characteristic information of an object, a set target value of characteristic information, or the like.

<<Details of Component>>

Details of the material, structure, and function of each will be described for the respective components of the device 100 described above.

(Irradiation Unit)

The irradiation unit 3 includes an irradiation optical system (not shown) and is a means for spreading pulsed light from a light source 15 with the irradiation optical system and then irradiating the object 1. The light source is capable of generating pulsed light of 5 nanoseconds to 50 nanoseconds. The light source is preferably a laser with which a large output can be obtained. However, this is not limiting, and it is also possible to use a light-emitting diode instead of a laser as the light source. For the laser to be used as the light source, various lasers such as a solid-state laser, gas laser, dye laser, or semiconductor laser can be applied. The laser to be used as the light source ideally has high output and can change the wavelength continuously. For example, a Nd:YAG pumped Ti:sa laser or alexandrite laser is advisable. In the light source, a plurality of single-wavelength lasers for different wavelengths may be held.

The irradiation unit 3 is a means for irradiating an object with pulsed light emitted from the light source 15, using the irradiation optical system. The irradiation optical system is typically an optical part such as a mirror that reflects light, a lens that spreads light, or a diffuser that diffuses light. Pulsed light from the light source 15 is guided to an object while being processed into a desired shape of irradiation light distribution with such an optical part. However, this is not limiting, and pulsed light from the light source 15 may be propagated using a waveguide such as optical fiber. For the optical part, anything with which the object 1 can be irradiated with pulsed light emitted from the light source 15 in a desired shape may be used. Pulsed light is preferably spread to a certain amount of area rather than be focused with a lens, in terms of safety for the object 1 and increasing the diagnosis region. With the irradiation unit 3, the pulsed light 11 may be radiated in different directions toward the object 1. In this case, it may be such that the irradiation unit is provided with a light irradiation unit scanning mechanism (not shown), and the light irradiation unit scanning mechanism irradiates the object 1 from different directions.

(Acoustic Wave Detection Unit)

The acoustic wave detection unit 4 is a means for detecting a photoacoustic wave generated on the surface or the inside of the object 1 and converting the photoacoustic wave to an electrical signal that is an analog signal. The acoustic wave detection unit 4 is also called a probe or transducer. For the acoustic wave detection unit 4, anything that can detect an acoustic wave signal, such as a transducer using the piezoelectric phenomenon, a transducer using resonance of light, or a transducer using a change in capacitance, may be used. The acoustic wave detection unit 4 is preferably formed by arranging a plurality of one-dimensionally or two-dimensionally arrayed acoustic wave detection elements and configured to be capable of mechanical scanning with an acoustic wave detection unit scanning mechanism. Accordingly, an acoustic wave can be detected in a plurality of positions, and the precision in acquiring characteristic information of an object improves. The acoustic wave detection unit 4 may be a single element on which an acoustic lens is focused, and the position of the generating source of an acoustic wave may accordingly be identifiable.

(Signal Processing Device)

The signal processing device 14 is a means for amplifying and converting to a digital signal an analog electrical signal input from the acoustic wave detection unit 4. The signal processing device 14 is typically configured of an amplifier, an A/D converter, a field programmable gate array (FPGA) chip, or the like. The signal processing device 14 may be capable of parallel processing of a plurality of signals simultaneously, in the case where there are a plurality of analog electrical signals input from the acoustic wave detection unit 4. Accordingly, the device 100 can shorten the time until acquisition of characteristic information of the object 1. When a photoacoustic wave is detected in a state where the relative positions of the object 1 and the acoustic wave detection unit 4 are the same, an analog electrical signal or digital signal that is the detection result may be integrated into one signal. A method of integration may be a mere addition of the signals, may be a method of adding of and dividing by the number of the signals, or may be weighting and adding of the respective signals.

(Calculation Processing Device)

The calculation processing unit 5 is a means for acquiring characteristic information of an object by performing calculation, image reconstruction, or the like of the light amount distribution. The calculation processing unit 5 typically includes a workstation and performs image reconstruction processing with software in which the image reconstruction processing or the like is programmed in advance. The calculation processing unit 5 may be provided with a plurality of pieces of hardware that separately perform respective processes for calculation of the characteristic information. The intensity of acoustic waves generated within an object is proportional to the Grüneisen coefficient dependent upon the temperature of the object. Therefore, the calculation processing unit 5 preferably converts the Grüneisen coefficient based on the temperature of the object 1 detected by a temperature sensor 16 described later and acquires characteristic information of the object 1 using the Grüneisen coefficient thus obtained.

(Object)

The object 1 does not constitute the device 100, but will be described herein for the purpose of illustration. The object 1 is a segment particularly of a human being in which a strain, arthritis, or the like often occurs, such as an ankle, knee, calf, thigh, shoulder, elbow, wrist, arm, back, or neck. However, this is not limiting, and the object 1 may be an animal.

(Absorber)

The absorber 9 does not constitute the device 100, but will be described herein for the purpose of illustration. The absorber 9 is particularly a place of blood accumulation in a blood vessel or segment that has swollen due to inflammation or of internal bleeding such as a bruise. The absorber 9 may be anything that absorbs the pulsed light 11.

(Water Bag)

The water bag 2 is configured to be capable of contacting the object 1. The water bag 2 is a plastic bag in which the solvent 12 is encapsulated for cooling or heating the object 1 through heat conduction between the water bag 2 and the object 1 by contacting the object 1. However, this is not limiting, and the water bag 2 may be anything that can encapsulate the solvent 12 and cause temperature propagation to the object 1.

The water bag 2 may be configured such that pressure can be applied to the object 1 via the solvent. In this case, a safety mechanism such as a safety valve that prevents a pressure of a certain level or higher from being applied may be provided for safety. The water bag 2 may be such that pressure can only be applied in a range of, for example, between 0 mmHg to 200 mmHg that is a predetermined pressure range, due to such a safety mechanism.

The acoustic wave detection unit 4 may receive photoacoustic waves via the water bag 2. In this case, it is desirable that the water bag 2 be configured of a material that provides adequate acoustic matching with the object 1.

The water bag 2 may be provided with the temperature sensor 16 (corresponding to a temperature detection unit) that can measure the temperature of the object 1 or the temperature of the solvent 12 within the water bag 2. The water bag 2 may be provided with a blood pressure meter capable of measuring the blood pressure of the object 1. Accordingly, the measurement result of the blood pressure meter can be input to the calculation processing unit 5 to adjust the temperature of the solvent 12 in accordance with the input result.

(Solvent)

The solvent 12 may formed by causing a superabsorbent polymer used in a refrigerant or the like to include water or may be formed only of water. However, this is not limiting, and the solvent 12 may be any medium that can transfer heat uniformly to the object 1. It is desirable that the solvent 12 be liquid or gas to which pressure can be applied with a pump or the like. The acoustic wave detection unit 4 may receive photoacoustic waves via the water bag 2 and the solvent 12. In this case, it is desirable that the solvent 12 be liquid that is low in acoustic decay rate and favorable in terms of acoustic matching with the water bag 2.

(Temperature Regulation Unit)

The temperature regulation unit 6 is formed of a heater that heats the solvent 12 and a Peltier element that cools the solvent 12. The temperature regulation unit 6 is a means for holding the temperature of the solvent 12 at a temperature designated by the control unit 8 based on the measurement result in the temperature sensor 16, by using the heater that heats the solvent 12 and the Peltier element that cools the solvent 12. The temperature regulation unit 6 may be provided with a safety mechanism that stops driving of the heater and the Peltier element, in the case where the temperature of the solvent 12 has exceeded a predetermined temperature range, e.g., in the case where it has become 42 degrees Celsius or higher or 0 degrees Celsius or lower. That is, accordingly, the heater and the Peltier element can be driven within a range of 0 degrees Celsius to 42 degrees Celsius.

In this embodiment, the water bag 2 and the temperature regulation unit 6 can be assumed as a temperature adjustment unit. The temperature adjustment unit according to the present invention is not limited to the above and may be anything that can change the temperature of the object 1.

(Control Unit)

The control unit 8 is a means for determining a sequence for causing the temperature regulation unit 6 to raise, hold, and lower the temperature of an object, based on characteristic information of the object acquired by the calculation processing unit 5 and a target value of characteristic information of the object set by the calculation processing unit. The control unit 8 controls the temperature regulation unit 6 based on the determined sequence.

A temperature control unit according to the present invention can control raising, holding, and lowering of the temperature of an object at a desired timing in accordance with the needs of therapy, based on characteristic information of the object. The temperature control unit according to the present invention may control any parameter of the temperature adjustment unit, as long as the temperature adjustment unit can be controlled to change the temperature of the object 1.

(Input Unit)

The input unit 7 is an input device for an operator to input a measurement parameter such as a target value of characteristic information, the temperature of the solvent 12, or time for which that is to be maintained. The input unit 7 may be a mouse or keyboard or may be a touch panel. This is not limiting, and the input unit 7 may be anything that is capable of inputting the information.

(Display Unit)

A display unit 13 is a monitor with which the progress of a sequence, the setting temperature of the temperature regulation unit 6, the actual temperature of the object 1 or the solvent 12, or characteristic information of an object is displayed and made visible. The display unit 13 may sound a buzzer that indicates the timing for detaching the water bag 2 from the object 1. The device 100 may be provided with a notification unit that is any means for notification of the timing for detaching an acoustic adjustment unit from an object.

An example of a temperature control method using the therapy device according to the present invention will be described.

For example, the control unit 8 first controls the temperature regulation unit 6 such that the temperature of the solvent 12 becomes a “temperature for lowering the temperature” to lower the temperature of the object 1. The calculation processing unit 5 acquires the amount of hemoglobin within the object. When the amount of hemoglobin acquired by the calculation processing unit 5 has reached a target value, the control unit 8 controls the temperature regulation unit 6 to maintain the temperature of the solvent 12 for a predetermined time. Accordingly, the temperature of the object is expected to be held constant when the amount of hemoglobin is at the target value. Subsequently, when the predetermined time has passed since the amount of hemoglobin has reached the target value, the control unit 8 controls the temperature regulation unit 6 such that the temperature of the solvent 12 becomes a “temperature for raising the temperature” to raise the temperature of the object 1. In a manner described above, the control unit 8 can control raising, holding, and lowering of the temperature of an object at a desired timing, based on characteristic information of the object.

The control unit 8 may further enhance the therapy effect by controlling the pressure applied to an object, in addition to temperature adjustment of the object, based on characteristic information of the object. In the case of the example described above, the control unit 8 may control the amount of inflow of the solvent 12 to the water bag 2 to apply pressure to the object, when the amount of hemoglobin has reached the target value and while the temperature of the object is being held constant.

### Embodiment 2

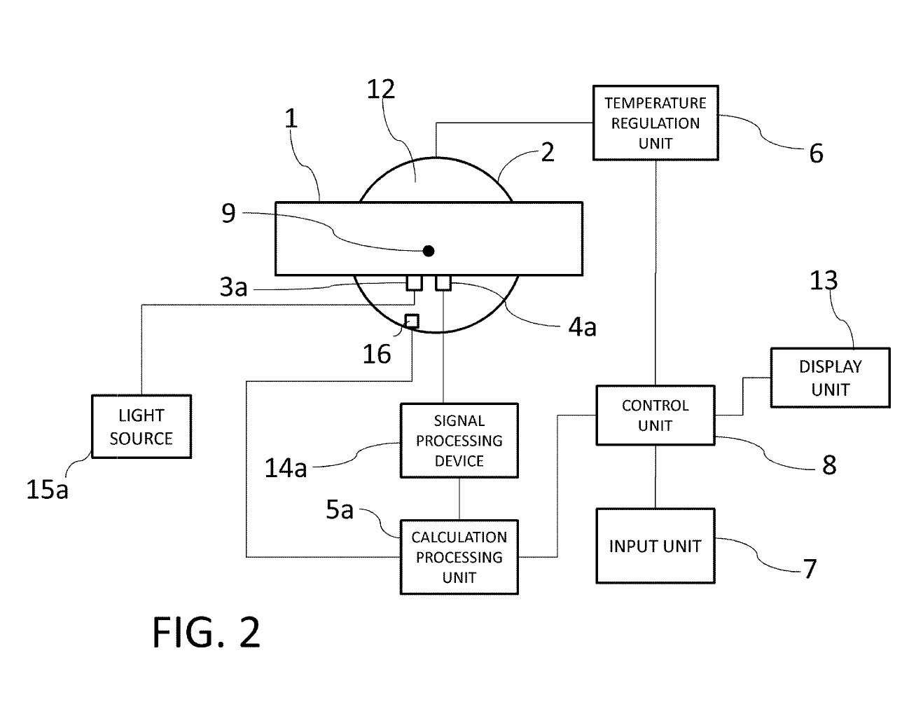

FIG. 2 is a block diagram showing Embodiment 2 of a therapy device according to an embodiment of the present invention. For portions corresponding to those in FIG. 1, the same reference numerals are assigned, and description will be omitted unless necessary.

The therapy device in Embodiment 2 is a therapy device 200 (hereinafter abbreviated as “device 200”) that uses propagation of diffusion light. The device 200 includes an irradiation unit 3a that irradiates the object 1 with single-wavelength light generated by a light source 15a and a light detection unit 4a that receives light reaching the light detection unit 4a as a result of the absorber 9 absorbing energy of a part of light and converts the received light to an analog electrical signal. Further, the device 200 includes a calculation processing unit 5a that calculates characteristic information of an object using an analog electrical signal and the water bag 2 containing the solvent 12 that is provided near the object 1 to contact the object 1 in order to change the temperature of the object 1. Further, the device 200 includes the temperature regulation unit 6 that changes the temperature of the object 1 by changing the temperature of the solvent 12 and the input unit 7 for an operator to input a target value of characteristic information of the object. Further, the device 200 includes the control unit 8 that controls the temperature regulation unit 6, based on characteristic information of the object 1 acquired by the calculation processing unit 5a and a set target value, and a display unit that displays the characteristic information of the object, the target value of the characteristic information, or the like. Since configurations other than the irradiation unit 3a, the light detection unit 4a, and the calculation processing unit 5a of this embodiment are similar to those in Embodiment 1, description is omitted herein, and only the configurations that are portions differing from the configurations in Embodiment 1 will be described.

(Irradiation Unit)

The irradiation unit 3a is formed of the light source 15a and an irradiation optical system. For the light source 15a, a light-emitting diode, laser, or the like may be used. The light source 15a is preferably that with which a large output can be obtained. For the laser to be used as the light source 15a, various lasers such as a solid-state laser, gas laser, dye laser, or semiconductor laser can be applied.

For measurement of the propagation of diffusion light, there are short pulse measurement, intensity-modulated light measurement, and continuous light measurement. It is desirable that the light source in the case of using short pulse measurement be capable of outputting pulsed light of several tens of picoseconds to 100 picoseconds. It is desirable that the light source 15a in the case of using intensity-modulated light measurement be capable of intensity modulation between 10 MHz and several hundred MHz. It is desirable that the light source in the case of using continuous light measurement be capable of outputting stable light continuously. It is desirable that the light source 15a be capable of outputting pulsed light, be formed of a plurality of single-wavelength laser diodes according to the wavelengths to be used for measurement, and be capable of outputting intensity-modulated light and continuous light. An irradiation optical system similar to that in Embodiment 1 may be used. With the irradiation unit 3a, light from the light source 15a may be radiated from different directions toward the object 1. In this case, it may be such that the irradiation unit 3a is provided with a light irradiation unit scanning mechanism (not shown), and the light irradiation unit scanning mechanism irradiates the object 1 from different directions.

(Light Detection Unit)

The light detection unit 4a is a means for detecting light that has propagated the surface and the inside of the object 1. With the light detection unit 4a, light is detected and converted to an electrical signal that is an analog signal. For the light detection unit 4a, a photomultiplier, an avalanche photodiode, photon counting, or the like may be used. With the light detection unit 4a, light that has propagated the surface and the inside of the object 1 may be focused with a lens and then detected, so that light is gathered to some degree and received.

(Signal Processing Device)

A signal processing device 14a is a means for amplifying and converting to a digital signal an analog electrical signal input from the light detection unit 4a. The signal processing device 14a is typically configured of an amplifier, an A/D converter, a field programmable gate array (FPGA) chip, or the like. The signal processing device 14a may be capable of parallel processing of a plurality of signals simultaneously, in the case where there are a plurality of analog electrical signals input from the light detection unit 4a. Accordingly, the device 200 can shorten the time until acquisition of characteristic information of the object 1.

(Calculation Processing Device)

The calculation processing unit 5a is a means for acquiring characteristic information of the object 1. The calculation processing unit 5a typically includes a workstation and performs image reconstruction processing with software in which the image reconstruction processing or the like is programmed in advance. In the case of a device utilizing a detection signal of light, characteristic information of an object to be acquired shows the absorption coefficient distribution, scattering coefficient distribution, and concentration distribution of a substance forming a tissue inside the object. The concentration distribution of a substance is, for example, oxygen saturation distribution, total hemoglobin concentration distribution, oxy/deoxy hemoglobin concentration distribution, or the like. It may be such that the degree of inflammation in an observed segment is estimated from the characteristic information, and such information is assumed as the characteristic information.

The calculation processing unit 5a may be provided with a plurality of pieces of hardware that separately perform respective processes for calculation of the characteristic information.

The intensity of light detected in this embodiment does not depend upon the temperature of the object 1 as in the intensity of acoustic waves generated in Embodiment 1. Therefore, the characteristic information does not need to be acquired based on the temperature of the object 1. That is, the device according to this embodiment does not need to be provided with a temperature sensor for detecting the temperature of the object 1 in order to improve the precision of the obtained characteristic information.

(Solvent)

The solvent 12 may formed by causing a superabsorbent polymer used in a refrigerant or the like to include water or may be formed of only water. Gas with large specific heat is also acceptable. However, this is not limiting, and the solvent 12 may be any liquid, gas, or the like that can transfer heat uniformly to the object 1. It is desirable that the solvent 12 be liquid or gas to which pressure can be applied with a pump or the like.

### Embodiment 3

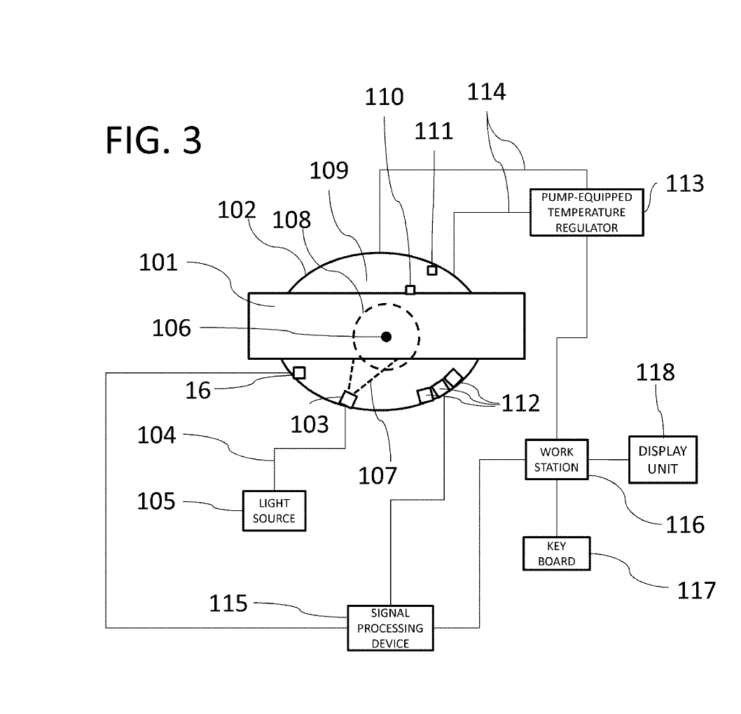

FIG. 3 is a block diagram showing Embodiment 3 of a therapy device according to an embodiment of the present invention. For portions corresponding to those in FIG. 1 or FIG. 2, the same reference numerals are assigned, and description is omitted unless necessary. In this embodiment, an object 101 is a human being. A therapy device 300 (hereinafter abbreviated as “device 300”) of this embodiment performs treatment by cooling, heating, or applying pressure to an affected area where a strain has occurred in a hamstring within the object 101. The device 300 determines the timing or period for raising, holding, or lowering the temperature of the object, from the amount of increase or decrease of blood within a blood vessel in the vicinity of the affected area that can be determined based on a photoacoustic image. The respective configurations of this embodiment will be described below.

A water bag 102 is wound around a thigh of the human being that is the object 101. The water bag 102 holds water that is a solvent 109 inside thereof. The water bag 102 is provided with a solvent thermometer 111 that can measure the temperature of the solvent 109 and an object thermometer 110 that can measure the temperature of the object 101, and is electrically coupled to a workstation 116 that is a control unit. The water bag 102 is provided with a drain 114 coupled with a pump-equipped temperature regulator 113. The solvent 109 subjected to temperature adjustment within the pump-equipped temperature regulator 113 flows into the water bag 102 via the drain 114 to accordingly transfer the temperature to the object 101. The pump-equipped temperature regulator 113 is coupled to the workstation 116 to be capable of exchanging signals, and the setting temperature is controlled by the workstation.

A light source 105 is a means for emitting pulsed light and is a YAG laser-induced Ti:sa laser. Pulsed light with a wavelength of 797 nm emitted from the light source 105 passes through an optical bundle fiber that is an optical waveguide 104 and enters an irradiation optical system 103.

The irradiation optical system 103 is a means for irradiating the object 101 with pulsed light 107 that is the entered light being spread. The irradiation optical system 103 is formed of a magnifying lens and a light amount measurement unit that measures the amount of light for a part of the pulsed light 107. The pulsed light 107 that has entered the object 101 scatters and propagates within the object 101 and is absorbed by blood that is an absorber 106 in the vicinity of an affected area to accordingly generate a photoacoustic wave 108.

A 2D array probe 112 is a means for receiving and converting to an analog electrical signal the generated photoacoustic wave 108 and sending the analog electrical signal to a signal processing device 115. The 2D array probe 112 is formed by a two-dimensional arrangement of 20×30 elements that are CMUTs with a center frequency of 3 MHz and a width of 1 mm.

The signal processing device 115 is a means for performing analog/digital conversion of the sent analog electrical signal to generate a digital signal.

In the workstation 116, a digital signal generated by the signal processing device 115 is input, and image reconstruction is performed with a universal back projection (UBP) method based on the input digital signal and an implemented program. The workstation 116 calculates the initial sound pressure based on the image reconstruction. The workstation 116 also calculates the light amount distribution inside the object 101 by solving a diffusion equation with a finite element method. The workstation 116 calculates an absorption coefficient distribution μₐ based on such initial sound pressure P₀ and light amount distribution 4, the Grüneisen constant Γ of the object 101, and formula (1).

P₀=Γμₐφ Formula (1)

The intensity of acoustic waves generated within an object is an intensity proportional to the Grüneisen coefficient dependent upon the temperature of the object. Therefore, the calculation processing unit 5 preferably converts the Grüneisen coefficient based on the temperature of the object 101 detected by the object thermometer 110 and acquires characteristic information of the object 101 using the Grüneisen coefficient thus obtained.

Further, the workstation 116 calculates the blood amount distribution or the hemoglobin amount distribution based on the absorption coefficient distribution μₐ that is the calculation result. With the light source 105, there may be at least two wavelengths of light that can be output. The workstation 116 may perform separate photoacoustic measurements based on respective wavelengths to acquire an absorption coefficient spectrum and calculate the oxygen saturation or the concentration distribution of a substance. The workstation 116 is coupled to be capable of exchanging signals with a keyboard 117 for an operator to input a target value of characteristic information of an object. The workstation 116 is coupled to be capable of exchanging signals with a monitor that is a display unit 118. The monitor that is the display unit 118 displays a sequence, the progress of processing executed by the sequence, the absorption coefficient distribution, blood amount distribution, or hemoglobin amount distribution in the vicinity of an affected area inside the object 101, the temperature of the solvent 109, the temperature of the object 101, or the like.

The workstation sets a target value of the amount of hemoglobin as characteristic information of an object. Although an example in which an operator sets a target value using the input unit has been shown herein, a method of setting a target value is not limited as such. For example, the workstation may acquire and set a target value stored in a storage device in accordance with the placement of an object.

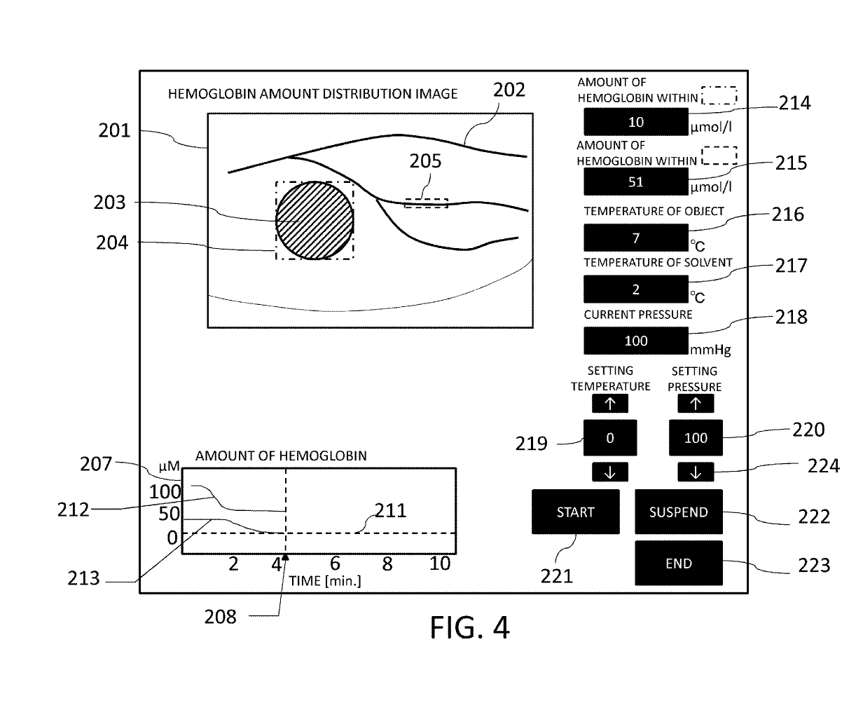

FIG. 4 is a diagram showing a screen of the display unit in the therapy device in Embodiment 3. In a hemoglobin amount distribution image 201 displayed in the display unit 118, blood 202 flowing in a blood vessel and an affected area 203 of internal bleeding due to strain or the like can be seen. In the display unit 118, a part of an imaged region (region of interest) is partitioned into designated regions 204 and 205 to allow display of a hemoglobin amount 214 in the designated region 204 and a hemoglobin amount 215 in the designated region 205. The display unit 118 can also display a target value 211 of the amount of hemoglobin. A display region 207 provided in the display unit 118 is capable of displaying a temporal shift 213 in the amount of hemoglobin in the designated region 204 and a temporal shift 212 in the amount of hemoglobin in the designated region 205. The display region 207 is capable of displaying the target value 211 of the amount of hemoglobin that is input using the keyboard 117. The abscissa in the display region 207 is the elapsed time from when a start button 221 has been pressed, and each shift is sequentially displayed even in a suspended state.

Further, the display screen of the display unit 118 is provided with an object temperature display unit 216 in which the current temperature of the object 101 is displayed, a solvent temperature display unit 217 in which the current temperature of the solvent 109 is displayed, and a pressure display unit 218 in which the current pressure displayed. The display screen of the display unit 118 is provided with a start button 221 for starting therapy, a suspend button 222 for suspending therapy, and an end button 223 for ending therapy. The display screen of the display unit 118 is provided with a setting temperature display unit 219 in which the current setting temperature is displayed, a setting pressure display unit 220 in which the current setting pressure is displayed, and a setting value change button 224 that allows the setting temperature or the setting pressure to be changed during suspension. The setting value change button 224 may be provided to allow a person to set the temperature while looking at the hemoglobin amount 214 or 215 in the designated region or the shift 207 in the amount of hemoglobin in the designated region.

The device 300 may be provided with a safety mechanism. For example, the safety mechanism may be as follows, in the case where the device 300 can calculate the oxygen saturation by irradiating the object 101 with light with two wavelengths. That is, the safety mechanism may stop applying pressure or stop lowering the temperature, when the amount of blood after light irradiation in the designated region 204 or 205 has decreased by 10% or more compared to that before the light irradiation.

With the therapy device having the configuration described above, an operator or control device can adjust the temperature of an object while referencing characteristic information or the current temperature of the object, with a target value of characteristic information being set in advance. Accordingly, appropriate temperature control is performed in accordance with the state of the object. Therefore, a favorable therapy effect can be expected.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

A new system configured of an appropriate combination of various techniques in the respective embodiments also falls within the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-265102, filed on Dec. 26, 2014, which is hereby incorporated by reference herein in its entirety.

What is claimed is:

1. A therapy device comprising:
a light source;
an irradiation unit configured to irradiate an object with light from the light source;
a reception unit configured to receive an acoustic wave generated and propagated within the object due to the light, and output the acoustic wave as an electrical signal;
a processing unit configured to acquire characteristic information of the object based on the electrical signal output from the reception unit;
a temperature adjustment unit configured to be capable of changing a temperature of the object; and
a temperature control unit configured to control the temperature adjustment unit based on the characteristic information.

2. The therapy device according to claim 1, further comprising a temperature detection unit configured to detect a temperature of the object, wherein
the processing unit is configured to acquire a Grüneisen coefficient of the object based on the temperature of the object detected by the temperature detection unit and to acquire the characteristic information based on the Grüneisen coefficient and the electrical signal.

3. A therapy device comprising:
a light source;
an irradiation unit configured to irradiate an object with light from the light source;
a light detection unit configured to receive light radiated from the irradiation unit and propagated within the object, and output the light as an electrical signal;
a processing unit configured to acquire characteristic information of the object based on the electrical signal output from the light detection unit;
a temperature adjustment unit configured to be capable of changing a temperature of the object; and
a temperature control unit configured to control the temperature adjustment unit based on the characteristic information.

4. The therapy device according to claim 1, wherein the processing unit acquires information on an amount of hemoglobin inside the object or information on an amount of blood inside the object, as the characteristic information of the object.

5. The therapy device according to claim 1, wherein the temperature adjustment unit is configured to be capable of holding a solvent and changing the temperature of the object by changing a temperature of the solvent, and
the temperature control unit is configured to control the temperature of the solvent based on the characteristic information.

6. The therapy device according to claim 5, wherein the temperature control unit is configured to control the temperature adjustment unit such that the temperature of the solvent falls within a predetermined range.

7. The therapy device according to claim 5, wherein the temperature control unit is configured to control the temperature adjustment unit such that the temperature of the solvent does not become 0 degrees Celsius or lower or 42 degrees Celsius or higher.

8. The therapy device according to claim 1, further comprising a setting unit configured to set a target value of the characteristic information, wherein
the temperature control unit is configured to control the temperature adjustment unit such that the characteristic information acquired by the processing unit approaches the target value.

9. The therapy device according to claim 8, further comprising an input unit configured to allow an operator to input information regarding the characteristic information, wherein
the setting unit is configured to set the target value based on the information regarding the characteristic information input by the input unit.

10. The therapy device according to claim 1, further comprising a setting unit configured to set a target value of the characteristic information, wherein
the temperature adjustment unit is configured to be capable of holding a solvent and changing the temperature of the object by changing a temperature of the solvent, and
the temperature control unit is configured to control the temperature adjustment unit to switch between raising, holding, and lowering of the temperature of the solvent when the characteristic information acquired by the processing unit has reached the target value.

11. The therapy device according to claim 1, further comprising a display unit configured to display the characteristic information of the object.

12. The therapy device according to claim 1, wherein the temperature adjustment unit is configured to be capable of applying pressure to the object based on the characteristic information.

13. The therapy device according to claim 3, wherein the processing unit acquires information on an amount of hemoglobin inside the object or information on an amount of blood inside the object, as the characteristic information of the object.

14. The therapy device according to claim 3, wherein the temperature adjustment unit is configured to be capable of holding a solvent and changing the temperature of the object by changing a temperature of the solvent, and
the temperature control unit is configured to control the temperature of the solvent based on the characteristic information.

15. The therapy device according to claim 14, wherein the temperature control unit is configured to control the temperature adjustment unit such that the temperature of the solvent falls within a predetermined range.

16. The therapy device according to claim 3, further comprising a setting unit configured to set a target value of the characteristic information, wherein
the temperature control unit is configured to control the temperature adjustment unit such that the characteristic information acquired by the processing unit approaches the target value.

17. The therapy device according to claim 16, further comprising an input unit configured to allow an operator to input information regarding the characteristic information, wherein
the setting unit is configured to set the target value based on the information regarding the characteristic information input by the input unit.

18. The therapy device according to claim 3, further comprising a setting unit configured to set a target value of the characteristic information, wherein
the temperature adjustment unit is configured to be capable of holding a solvent and changing the temperature of the object by changing a temperature of the solvent, and
the temperature control unit is configured to control the temperature adjustment unit to switch between raising, holding, and lowering of the temperature of the solvent when the characteristic information acquired by the processing unit has reached the target value.

19. The therapy device according to claim 3, further comprising a display unit configured to display the characteristic information of the object.

20. The therapy device according to claim 3, wherein the temperature adjustment unit is configured to be capable of applying pressure to the object based on the characteristic information.
